(19) United States
(12) Patent Application Publication
(10) Pub. No.: US 2019/0307484 A1
(43) Pub. Date: Oct. 10, 2019

(54) LUMBAR SYRINGE GUIDE ASSEMBLY

(71) Applicant: Pain Away Solutions, LLC, Merced, CA (US)

(72) Inventor: Gabriel Garcia Diaz, Merced, CA (US)

(21) Appl. No.: 16/380,777

(22) Filed: Apr. 10, 2019

Related U.S. Application Data
(60) Provisional application No. 62/655,666, filed on Apr. 10, 2018.

(51) Int. Cl. A61B 17/34; A61M 5/42

(52) U.S. Cl. CPC A61B 17/3403; A61M 5/427; A61B 2017/3411; A61B 2017/3407; A61B 17/3401

(57) ABSTRACT

A medical assembly to assist physicians with holding and guiding multiple needles for medical procedures may include a targeter having a frame having two elongate parallel supports attached to one another by at least one adjustable central arm; and a plurality of ports attached to each elongate support, wherein the ports are each sized to accommodate a syringe needle. The ports may be attached to their respective support at an angle of 15°. Moreover, the supports may each include a fastener to removably secure the targeter to a patient.

### REFERENCE TO PENDING PRIOR PATENT APPLICATION

This patent application claims benefit of pending prior U.S. Provisional Patent Application Ser. No. 62/655,666, filed Apr. 10, 2018 by Gabriel Garcia Diaz for LUMBAR SYRINGE GUIDE ASSEMBLY (Attorney's Docket No. PAIN-1 PROV: GARCIA-DIAZ.G-LZ.001PP), which patent application is hereby incorporated herein by reference.

### BACKGROUND

The embodiments described herein relate generally to medical devices, and more particularly, to a lumbar syringe guide assembly.

Surgeons, such as spine surgeons regularly use injections to treat patients with acute and chronic pain. A large number of patients may also require fluoroscopic guided spinal and joint fluoroscopic guided injections.

Currently, physicians handle one syringe at a time, wherein they need to find the correct location and introduce an angle of 15 degrees for insertion of the syringe. Having to handle each syringe independently leaves room for error, particularly when it comes to precision of the insertion. Moreover, because each syringe has to be handled separately, this adds time and radiation exposure to the patient and the healthcare staff involved in the care of the patient.

Therefore, what is needed is a device to assist a physician in holding and guiding multiple needles with syringes simultaneously, while ensuring the accuracy of the insertion angle and location.

### SUMMARY

Some embodiments of the present disclosure include a medical assembly to assist physicians with holding and guiding multiple needles for medical procedures. The medical assembly may include a targeter having a frame having two elongate parallel supports attached to one another by at least one adjustable central arm; and a plurality of ports attached to each elongate support, wherein the ports are each sized to accommodate a syringe needle. The ports may be attached to their respective support at an angle of 15°. Moreover, the supports may each include a fastener to removably secure the targeter to a patient.

### BRIEF DESCRIPTION OF THE FIGURES

Having thus described the invention in general terms, reference is now made to the accompanying figures, which show different views of different example embodiments.

FIGS. 1 and 2 are schematic views of a medical assembly formed in accordance with the present invention.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used as a device to hold and guide multiple needles with syringes simultaneously and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

1. Frame

2. Ports

3. Fastener

The various elements of the device of the present invention may be related in the following exemplary fashion. It is not intended to limit the scope or nature of the relationships between the various elements and the following examples are presented as illustrative examples only.

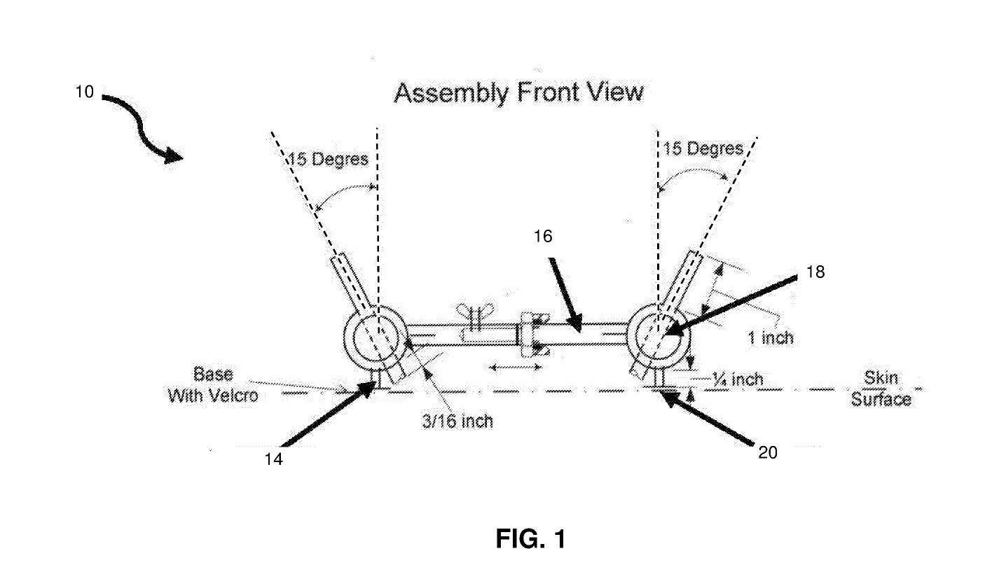

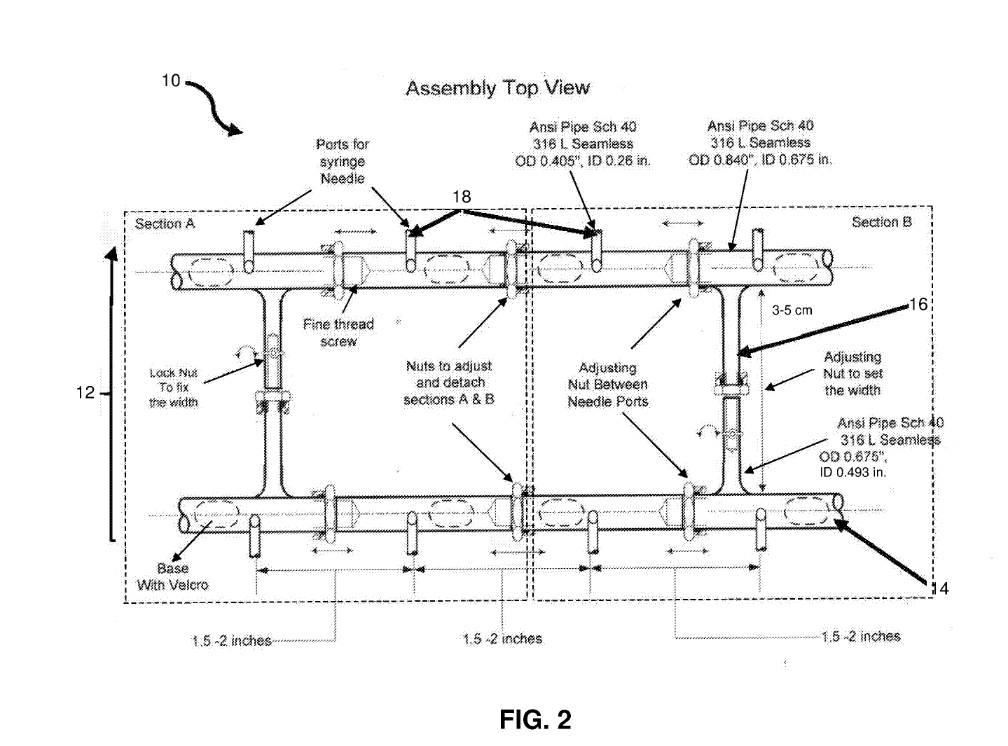

By way of example, and referring to FIGS. 1 and 2, some embodiments of the invention include a medical assembly to assist physicians with holding and guiding multiple needles for medical procedures, such as spinal procedures, the assembly comprising a targeter 10 comprising a frame 12 having two elongate parallel supports 14 attached to one another by at least one adjustable central arm 16, and a plurality of ports 18 attached to each elongate support 14, wherein the ports 18 are each sized to accommodate a syringe needle. In embodiments, each of the elongate supports 14 may have a fastener 20, such as Velcro brand hook and loop fastener, attached to a bottom surface thereof, such that the frame 12 may be secured in place by engaging the fastener 20 with a complementary fastener removably attached (such as adhesively attached) to a patient's body.

In some embodiments, multiple assemblies of the present disclosure may be combined by attaching an end of a first elongate support 14 to an end of an elongate parallel support 14 from a separate assembly. The elongate supports 14 may be attached using any suitable fastener, such as by using a screw.

As described above, the elongate parallel supports 14 in an assembly are attached to one another by at least one central arm 16. The central arm 16 may have an adjustable length to accommodate various spinal width and disk clearances. For example, the central arm 16 may be a telescoping tube with a lock nut, wherein the lock nut functions to lock the central arm 16 at the required or desired length, thus locking the assembly at the necessary width.

As shown in the figures, the ports 18 comprise an internal guide hollow tube that is designed to hold the needle. Each port 18 may be attached to its respective support 14 at a specific angle, such as at a 15° angle.

The elongate supports may have a fastener, such as Velcro brand hook and loop fastener, attached to a bottom surface thereof such that the frame may be secured in place. Each of the ports may be attached to the support at a 15 degree angle.

To use the medical assembly of the present disclosure, a physician may position the assembly and confirm the placement under fluoroscopic guidance. The medical assembly may be secured to the patient's body using the fastener 20. Once the placement is confirmed, all necessary syringes may be prepared and introduced into the hollow tubes within the ports 18. Thus, all required syringes may be placed at the same time, saving time and radiation and, therefore, increasing efficiency and precision simultaneously. The medical assembly may be used in various medical procedures including spinal applications currently performed for pain management including cervical, thoracic, lumbar spine medial branch blocks, facet blocks, thoracic, lumbar transforaminal epidural blocks, and sacroiliac joint blocks.

While the embodiments shown in the Figures are directed to assemblies that are manually placed and adjusted. However, it is envisioned that a software and navigation program with robotic guided injections may be added to the assembly. This addition may further improve precision, decrease radiation exposure, and increase injection speed.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claim.

What is claimed is:

1. A medical assembly to assist physicians with holding and guiding multiple needles for medical procedures, the assembly comprising:
a targeter comprising a frame having two elongate parallel supports attached to one another by at least one adjustable central arm; and
a plurality of ports attached to each elongate support, wherein the ports are each sized to accommodate a syringe needle.
